(19) United States
(12) Patent Application Publication
(10) Pub. No.: US 2005/0171374 A1
(43) Pub. Date: Aug. 4, 2005

(54) Preparation of levulinic acid esters from alpha-angelica lactone and olefins; use of ester compositions as fuel additives

(76) Inventor: Leo Ernest Manzer, Wilmington, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 10/768,276

(22) Filed: Jan. 30, 2004

(51) Int. Cl. C12P 7/62; C07C 69/66

(52) U.S. Cl. 560/190; 435/135

(57) ABSTRACT

This invention relates to a process for producing levulinic acid esters from α-angelica lactone and olefins, the compositions produced therefrom, and the uses thereof. This invention also relates to a process for producing a mixture of levulinic acid and formic acid esters from α-angelica lactone plus formic acid and olefins, or from biomass and olefins. This invention also relates to the uses of these esters as fuel and fuel additives for gasoline fuel, diesel fuel, and biofuel.

### FIELD OF INVENTION

This invention relates to a process for producing levulinic acid esters from α-angelica lactone and olefins, and the compositions produced therefrom. This invention also relates to the use of the mixture of these esters as fuel and fuel additives for gasoline fuel, diesel fuel, and biofuel. This invention also relates to a process for producing a mixture of levulinic acid and formic acid esters from α-angelica lactone plus formic acid and olefins, or from biomass and olefins, as well as the compositions produced therefrom and the uses thereof.

### TECHNICAL BACKGROUND OF THE INVENTION

“Fuel additives” are substances that can improve the fuel efficiency of an engine, for example, as measured by the octane number, the cetane number or any other index suited to measure the efficiency of a particular fuel. A fuel additive may also perform the function of lubricating, cleaning and stabilizing the fuel and may improve performance, economy, and injector life, reduce emissions, reduce smoke related to an engine, help eliminate tank draining, lower a gel point of the fuel or provide a clean burning fuel that can inhibit polluting agents in emissions. The compositions produced by the processes of the invention are intended for use as desirable fuel additives.

“Oxygenates” is a commonly referred to group of chemical compounds that raise the oxygen content of gasoline. Oxygen helps gasoline burn more completely, reducing harmful tailpipe emissions from motor vehicles. In one respect, the oxygen dilutes or displaces gasoline components such as aromatics (e.g., benzene), and sulfur. Additionally, it optimizes oxidation during combustion. Most gasoline suppliers meet the oxygen content requirements of the different clean fuel programs by adding oxygenate fuel additives, most commonly methyl tertiary-butyl ether (hereinafter referred to as MTBE), to gasoline blend stocks. Recently, various environmental protection agencies have begun raising concerns regarding the detection of MTBE in surface and ground water.

A need exists to develop esters with low water solubility that could be used to meet governmental oxygen requirements for gasoline and oxy-gasoline fuels. These low solubility esters would have a reduced solubility in surface and subsurface water and could therefore reduce the impact on such waters from spills and emissions of oxygenated fuels. It would also be desirable for MTBE replacements, including these esters, to have other favorable properties such as low ozone formation potential and a low rubber seal-swelling tendency. U.S. 2003/0233011A1 describes a process for producing levulinic acid and formic acid esters from biomass. Levulinic acid and formic acid produced by the hydrolysis of biomass are reacted with olefins to produce levulinate and formate esters.

The present invention relates to improved processes for producing levulinic acid esters from α-angelica lactone and olefins and to novel products produced. The α-Angelica lactone for use in this process may be derived from biomass. The invention described herein also relates to use of the esters as oxygenates produced in the processes in automotive gasoline used in internal combustion engines, as oxygenates in diesel engines, as additives to biofuel and also directly as fuel.

### SUMMARY OF THE INVENTION

Described herein is a process for preparing levulinic acid esters (II) from a reaction of α-angelica lactone with olefins. The process comprises:
(a) contacting α-angelica lactone (I) with at least one olefin in the presence of water and an acid catalyst, optionally in the presence of a water immiscible solvent as shown in the equation below: 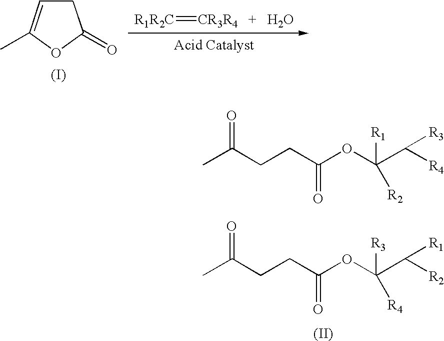
   wherein:
(i) R₁, R₂, R₃ and R₄ are independently selected from the group consisting of hydrogen, C₁-C₁₀ unsubstituted or substituted alkyl, C₁-C₁₀ unsubstituted or substituted alkenyl and C₃-C₂₀ unsubstituted or substituted cycloalkyl, wherein optionally any two or more of R₁, R₂, R₃, and R₄ can together form a cyclic or bicyclic alkyl group, wherein the unfilled valences in α-angelica lactone and the levulinic acid esters are hydrogen, and wherein the total number of carbons in the olefin is not more than twenty two; and
(ii) the levulinic acid esters (II) comprise 10% to 100% of the products formed; and
(b) optionally recovering said levulinic acid esters.

Described herein is also a process for preparing a mixture of levulinic acid esters and formic acid esters from a reaction of α-angelica lactone plus formic acid with olefins that comprises:
(a) contacting α-angelica lactone (I) plus formic acid with at least one olefin in the presence of water and an acid catalyst, optionally in the presence of a water immiscible solvent: 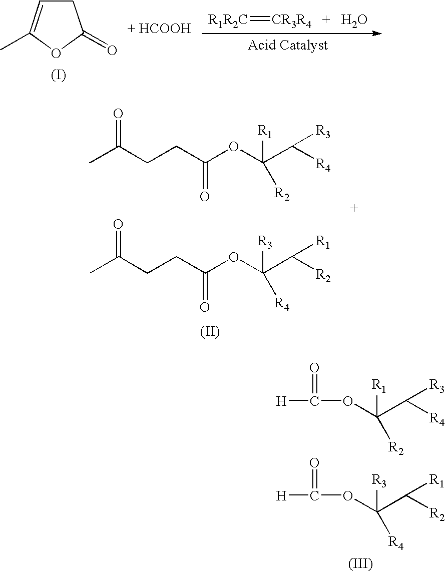
   wherein:
(i) R₁, R₂, R₃ and R₄ are independently selected from the group consisting of hydrogen, C₁-C₁₀ unsubstituted or substituted alkyl, C₁-C₁₀ unsubstituted or substituted alkenyl and C₃-C₂₀ unsubstituted or substituted cycloalkyl, wherein optionally any two or more of R₁, R₂, R₃, and R₄ can together form a cyclic or bicyclic alkyl group, wherein the unfilled valences in α-angelica lactone, the levulinic acid esters and the formic acid esters are hydrogen, and wherein the total number of carbons in the olefin is not more than twenty two; and
(ii) the levulinic acid esters (II) and formic acid esters (III) together comprise 10% to 100% of the products formed; and
(b) optionally recovering said levulinic acid esters and formic acid esters.

The acid catalyst useful for the above processes of the invention preferably comprises from about 0.01% to about 50% by weight of the reactants. The temperature for the processes of the invention described above is preferably from about 0° C. to about 300° C. The pressure for the processes of the invention described above preferably is from about 0.1 MPa to about 13.8 MPa. The molar ratio of water to α-angelica lactone is from about 100 to 0.05. Levulinic acid may be produced in the processes of the invention; levulinic acid may be converted to α-angelica lactone and recycled back to the reactions above for conversion to levulinic acid esters.

The reactions of the invention described above are catalyzed by an acid catalyst that preferably has a pKa of less than 4 or that is a heterogeneous catalyst having an Hₒ of less than or equal to 2. The acid catalyst is preferably selected from the group consisting of inorganic acids, organic sulfonic acids, heteropolyacids, perfluoroalkyl sulfonic acids, metal salts thereof, mixtures of metal salts, and combinations thereof. The acid catalyst may also be selected from the group consisting of zeolites; CV-3020 zeolite; fluorinated alumina; acid-treated silica; acid-treated silica-alumina; acid-treated titania; acid-treated zirconia; heteropolyacids supported on zirconia, titania, alumina, silica; and combinations thereof. The acid catalyst may also be selected from the group consisting of metal sulfonates, metal sulfates, metal trifluoroacetates, metal triflates, and mixtures thereof; mixtures of salts with their conjugate acids, zinc tetrafluoroborate, and combinations thereof.

The α-angelica lactone and formic acid useful for the processes of the invention may be derived from biomass by a process comprising:
(a) contacting biomass with water in the presence of an acid catalyst in one or more reactors, wherein:
   (i) the initial biomass concentration comprises about 1% to about 50% biomass solids by weight,
   (ii) the acid catalyst comprises about 1% to about 30% acid by weight,
   (iii) the pressure of the reaction is about 0.69 MPa to about 13.8 MPa,
   (iv) the temperature of the reaction is about 150° C. to about 300° C., and
   (v) the time for the reaction is about 3 seconds to about 180 minutes;
      to form a first reaction mixture comprising levulinic acid, formic acid and solids;
(b) reducing the pressure of said first reaction mixture such that an aqueous stream of gaseous formic acid is produced to form a second reaction mixture comprising levulinic acid and solids;
(c) condensing said gaseous formic acid produced in step (b) to form a third reaction mixture comprising formic acid;
(d) optionally removing said solids from said second reaction mixture and recovering said levulinic acid; and
(e) converting said levulinic acid to α-angelica lactone by using vacuum distillation to form a fifth reaction mixture.

The α-angelica lactone and formic acid useful for the processes of the invention may also be derived from biomass by a process comprising:
(a) contacting biomass with water in the presence of an acid catalyst in a reactor, wherein:
   (i) the initial biomass concentration comprises about 1% to about 50% biomass solids by weight,
   (ii) the acid catalyst comprises about 1% to about 10% acid by weight,
   (iii) the pressure of the reaction is about 0.69 MPa to about 13.8 MPA,
   (iv) the temperature of the reaction is about 200° C. to about 250° C., and
   (v) the time for the reaction is about 5 seconds to about 120 minutes;
   to form a first reaction mixture comprising
   hydroxymethylfurfural, optionally furfural and other reaction intermediates, and solids;
(b) optionally transferring said first reaction mixture to a second reactor wherein:
   (vi) the acid concentration is from about 2% to about 10%,
   (vii) the temperature of the reaction is from about 150° C. to about 210° C., and
   (viii) the time for the reaction is from about 10 minutes to about 60 minutes;
   to form a second reaction mixture comprising levulinic acid, formic acid, optionally furfural and other reaction intermediates, and solids;
(c) removing said formic acid and optionally said furfural from said second reaction mixture by condensation to form a third reaction mixture comprising formic acid and optionally furfural and a fourth reaction mixture comprising levulinic acid, optionally other reaction intermediates, and solids;
(d) optionally removing said solids from said fourth reaction mixture and recovering said levulinic acid;
(e) converting said levulinic acid to α-angelica lactone by using vacuum distillation to form a fifth reaction mixture; and
(f) optionally removing furfural from said third reaction mixture by distillation or liquid-liquid separation to form a sixth reaction mixture comprising formic acid.

Described herein are also the compositions of the esters formed by the reactions of 1) α-angelica lactone with olefins, 2) α-angelica lactone plus formic acid with olefins and 3) biomass conversion to levulinic acid esters and to mixtures of levulinic acid :and formic acid esters. These compositions are useful as fuel, an oxygenate for gasoline, an octane number-enhancing agent for gasoline, an oxygenate for diesel, a cetane number-enhancing agent for diesel or a fuel additive for biofuel. Gasoline, diesel or biofuel compositions comprising from 1% to 90% by volume of the composition as esters are also described. Processes are also described for producing fuel additives, gasoline, diesel fuel or biofuel by using the processes of the invention.

### DETAILED DESCRIPTION OF THE INVENTION

The present invention relates to the use of esters with low water solubility as fuel additives. Described herein is a method for producing fuel additives comprising a mixture of levulinic acid esters derived from α-angelica lactone for use as oxygenates. Described herein additionally is a method for producing a mixture of both levulinic and formic acid esters from α-angelica lactone plus formic acid for use as oxygenates. Besides being used as oxygenate additives for diesel fuel, gasoline fuel and biofuel, the esters of the invention can also be used as octane number-enhancing agents for gasoline, and as cetane number-enhancing agents in diesel fuels. The reaction mixture of esters of the present invention can also be directly used as 100% fuel. More specifically, the process of the present invention describes a method for producing ester-based fuel additives by the reaction of olefins with α-angelica lactone, or by the reaction of olefins with α-angelica lactone plus formic acid.

α-Angelica lactone as used herein means a compound having the following formula:

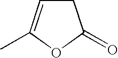

“Levulinic acid” as used herein means a compound having the following formula, or a salt thereof:

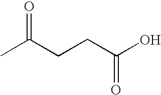

“Formic acid” as used herein means a compound having the following formula, or salt thereof:

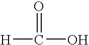

“Olefin” herein means a compound having the following general formula: 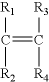
wherein R₁, R₂, R₃ and R₄are independently selected from the group consisting of hydrogen and linear, branched or cyclic alkyl hydrocarbyl groups, including C₁-C₁₀ unsubstituted or substituted alkyl, C₁-C₁₀ unsubstituted or substituted alkenyl and C₃-C₂₀ unsubstituted or substituted cycloalkyl, wherein optionally any two or more of R₁, R₂, R₃, and R₄ can together form a cyclic or bicyclic alkyl group, and wherein the total number of carbons is not more than twenty two.

A preferred olefin for this invention is one wherein R₁, R₂, R₃, and R₄ are independently hydrogen or an alkyl group, and wherein the total number of carbons in the olefin is not more than ten.

“Levulinic acid ester” of the invention means an ester having one of the formulas exemplified below, wherein R₁, R₂, R₃ and R₄ are independently selected from the group consisting of hydrogen, C₁-C₁₀ unsubstituted or substituted alkyl, C₁-C₁₀ unsubstituted or substituted alkenyl and C₃-C₂₀ unsubstituted or substituted cycloalkyl, wherein optionally any two or more of R₁, R₂, R₃, and R₄ can together form a cyclic or bicyclic alkyl group, wherein the unfilled valences in the levulinic acid esters are hydrogen, and wherein the total number of carbons in the levulinic acid esters is not more than twenty seven:

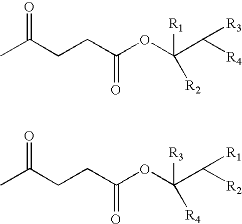

“Formic acid ester” of the invention means an ester having one of the formulas exemplified below, wherein R₁, R₂, R₃ and R₄ are independently selected from the group consisting of hydrogen, C₁-C₁₀ unsubstituted or substituted alkyl, C₁-C₁₀unsubstituted or substituted alkenyl and C₃-C₂₀ unsubstituted or substituted cycloalkyl, wherein optionally any two or more of R₁, R₂, R₃, and R₄ can together form a cyclic or bicyclic alkyl group, wherein the unfilled valences in the formic acid esters are hydrogen, and wherein the total number of carbons in the formic acid esters is not more than twenty three:

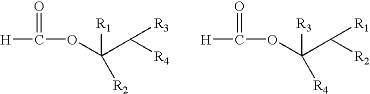

Preferred levulinic acid esters and formic acid esters of the invention are those wherein R₁, R₂, R₃, and R₄ are independently hydrogen or an alkyl group, and wherein the total number of carbons in the ester is not more fifteen for levulinic acid esters and eleven for formic acid esters.

By the term “sugar” is meant a five-carbon pentose sugar or a six-carbon hexose sugar.

By the term “acid catalyst” is meant a substance which has the ability to donate protons as defined by Bronsted, or a substance which can form a covalent bond with an atom, molecule or ion that has an unshared electron pair as defined by Lewis. Any known acid catalysts can be used for the process of the present invention. A further definition of acid catalysts and how to determine if a particular substance is acidic is explained in Tanabe, K., Catalysis: Science and Technology, Vol. 2, pg. 232-273, ed. Anderson, J. and Boudart, M., Springer-Verlag, N.Y., 1981.

By the term “biofuel” is meant either a 100% biodiesel or a mixture comprising biodiesel and regular petroleum-based diesel from a refinery. For example, B20 is a mixture of 20% biodiesel based on vegetable oil, and 80% regular diesel. Biodiesel is a product of esterification of oils such as palm, canola, tallow, corn, and soy, with methanol.

By the term “octane number” is meant an empirical rating of the anti-knock quality of a fuel. “Knock” is caused by secondary ignition of fuel unburned after normal spark ignition, which gives rise to a fast moving flame front in an automobile's engine cylinder. Pressure waves are setup, which vibrate against the cylinder walls giving rise to a “knocking” sound. This feature of fuel is undesirable because it accelerates wear in the engine bearings and causes overheating in the cylinders. The tendency of the fuel to knock increases as the compression ratio increases. Certain fuels have better anti-knock characteristics than others because of their molecular structure, branched structures having better characteristics. On the arbitrary octane scale, iso-octane (C₈H₁₈) is given an octane value of 100; n-heptane(C₇H₁₆), is given a value of zero. The octane number of a fuel is determined by comparing its performance in a standard spark-ignition engine with the performance of various mixtures of iso-octane and n-heptane. The behavior of the fuel is carefully matched by a known mixture of iso-octane and n-heptane. The percentage of isooctane in this mixture is then taken as the octane number of the fuel.

The “cetane number” is used to evaluate fuels used in compression-ignition (diesel) engines and is analogous to octane number. Cetane (n-hexadecane, C₁₆H₃₄) is designated 100 and alpha-methyl-naphthalene(C₁₁H₁₀) as zero, so that the cetane number of a fuel is the proportion of the cetane in the mixture of these having the same ignition delay after injection of the fuel as the test fuel.

As used herein, the term “biomass” refers to a primarily carbohydrate-containing material. Biomass can also refer to a polysaccharide-containing material. It can also refer to a cellulose-, hemicellulose-, or lignocellulose-containing material. Biomass is commonly obtained from, for example, wood, plants, residue from agriculture or forestry, organic component of municipal and industrial wastes, primary sludges from paper manufacture, waste paper, waste wood (e.g., sawdust), agricultural residues such as corn husks, corn cobs, rice hulls, straw, bagasse, starch from corn, wheat oats, and barley, waste plant material from hard wood or beech bark, fiberboard industry waste water, bagasse pity, bagasse, molasses, post-fermentation liquor, furfural still residues, aqueous oak wood extracts, rice hull, oats residues, wood sugar slops, fir sawdust, naphtha, corncob furfural residue, cotton balls, rice, straw, soybean skin, soybean oil residue, corn husks, cotton stems, cottonseed hulls, starch, potatoes, sweet potatoes, lactose, waste wood pulping residues, sunflower seed husks, hexose sugars, pentose sugars, sucrose from sugar cane and sugar beets, corn syrup, hemp, and combinations of the above.

The invention described herein provides a process for preparing levulinic acid esters (II) from α-angelica lactone. The process comprises contacting α-angelica lactone (I) with at least one olefin (R₁R₂C═CR₃R₄) in the presence of water and an acid catalyst, and optionally in the presence of a water immiscible solvent as shown in the following Reaction (A):

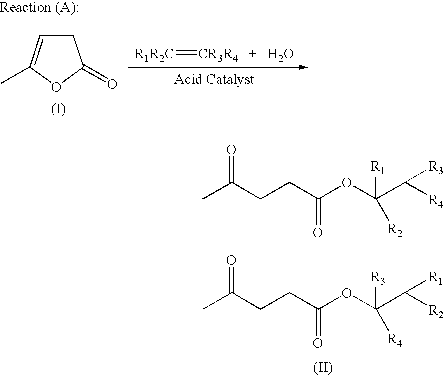

In the present invention, R₁, R₂, R₃ and R4 are independently selected from the group consisting of hydrogen, C₁-C₁₀ unsubstituted or substituted alkyl, C₁-C₁₀ unsubstituted or substituted alkenyl and C₃-C₂₀ unsubstituted or substituted cycloalkyl, wherein optionally any two or more of R₁, R₂, R₃, and R₄ can together form a cyclic or bicyclic alkyl group, wherein the unfilled valences in α-angelica lactone and the levulinic acid esters are hydrogen, and wherein the total number of carbons in the olefin is not more than twenty two.

Typical olefins useful for the reaction include ethylene, propylene, butene, pentene, hexene, cyclohexene, etc. Mixtures of olefins may also be used.

The alkyl hydrocarbyl olefin of the present invention may comprise more than one double bond. In those instances where the olefin contains more than one double bond, the products may comprise a mixture of esters formed by the reaction of α-angelica lactone with one or more of the double bonds within the olefin.

Optionally, the olefins may be added in the presence of a water-immiscible hydrocarbon solvent. Water-immiscible solvents that are suitable for the process of the invention include aromatic hydrocarbons, such as toluene, xylene, and ethyl benzene. Aliphatic hydrocarbons such as n-hexane, n-heptane, and iso-octane are also suitable. Also, in certain instances, water-immiscible materials such as cycloaliphatic hydrocarbons (e.g. cyclohexane) and pelargonic acid and the like, may also be used. In general, any material which is substantially water-immiscible and which will extract a good portion of the esters but will not extract appreciable quantities of the acid may be employed in this step of the process. Of these extractants, iso-octane is an especially suitable immiscible solvent as it is readily available, chemically inert, easily recovered, and can be used as an additive to the fuel. Solvent mixtures may also be used.

After the addition of the water-immiscible solvent, an aqueous phase and an organic phase are allowed to layer out and the two phases are separated for further processing, for example, by decantation.

As a result of this procedure, levulinic acid esters are extracted from the organic phase. The separated organic phase comprising the ester mixture and the organic solvent can be added as a fuel additive to hydrocarbon fuel.

Alternatively, the hydrocarbon phase from the above process can also be preferably distilled at atmospheric pressure to recover the immiscible solvent. The residue from this operation is distilled, preferably under vacuum, to yield the esters of levulinic acid.

Water is required for Reaction (A). A molar ratio of about 100 to about 0.05 of water to α-angelica lactone is preferred. A molar ratio of about 2 to 1 is further preferred. A molar ratio of about 1 to 1 is further preferred.

Acid catalysts that may be employed in this process of the invention include inorganic acids such as sulfuric acid, phosphoric acid, hydrochloric acid, and nitric acid, as well as mixtures thereof. Organic acids such as para-toluene sulfonic acid, triflic acid, trifluoroacetic acid and methanesulfonic acid may also be used. Moreover, ion exchange resins in the acid form may also be employed. Hence, any type of acid catalyst known in the art may be employed.

Fluorinated sulfonic acid polymers can also be used as acid catalysts for the process of the present invention. These acids are partially or totally fluorinated hydrocarbon polymers containing pendant sulfonic acid groups, which may be partially or totally converted to the salt form. One particularly suitable fluorinated sulfonic acid polymer is Nafion® perfluorinated sulfonic acid polymer, (E.I. du Pont de Nemours and Company, Wilmington, Del.). One preferred form is Nafion® Super Acid Catalyst, a bead-form strongly acidic resin which is a copolymer of tetrafluoroethylene and perfluoro-3, 6-dioxa-4-methyl-7-octene sulfonyl fluoride, converted to either the proton (H⁺), or the metal salt form.

A soluble acid catalyst may also be used during the method of the invention. Suitable soluble acids include, those acid catalysts with a pKa less than about 4, preferably with a pKa less than about 2, including inorganic acids, organic sulfonic acids, heteropolyacids, perfluoroalkylsulfonic acids, and combinations thereof. Also suitable are metal salts of acids with pKa less than about 4, including metal sulfonates, metal sulfates, metal trifluoroacetates, metal triflates, and mixtures thereof, including mixtures of salts with their conjugate acids. Specific examples of suitable acids include sulfuric acid, fluorosulfonic acid, phosphoric acid, p-toluenesulfonic acid, benzenesulfonic acid, phosphotungtstic acid, phosphomolybdic acid, trifluromethanesulfonic acid, 1,1,2,2-tetrafluroethanesulfonic acid, 1,2,3,2,3,3-hexapropanesulfonic acid, bismuth triflate, yttrium triflate, ytterbium triflate, neodymium triflate, lanthanum triflate, scandium triflate, zirconium triflate, and zinc tetrafluoroborate.

The acid catalyst is preferably used in an amount of from about 0.01% to about 50% by weight of the reactants, although the concentration of acid catalyst may exceed 50% if the reaction is run in continuous mode using a packed bed reactor. A preferred range is 0.25% to 5% by weight of the reactants unless the reaction is run in continuous mode using a packed bed reactor.

Heterogeneous acid catalysts can also be used during the process of the invention. Suitable heterogeneous acids are, for example, zeolites, CBV-3020 zeolite, fluorinated alumina, acid-treated silica, acid treated silica-alumina, acid treated clays, heterogeneous heteropolyacids and sulfated zirconia. The acid catalyst can also be selected from the group consisting of sulfuric acid-treated silica, sulfuric acid-treated silica-alumina, acid-treated titania, acid-treated zirconia, heteropolyacids supported on zirconia, heteropolyacids supported on titania, heteropolyacids supported on alumina, heteropolyacids supported on silica, and combinations thereof. Suitable heterogeneous acid catalysts include those having an Ho of less than or equal to 2.

A temperature range of from about 0° C. to about 300° C. is preferred for the process of the invention. A temperature range of from about 100 to about 200 is further preferred.

In the process of the invention, a pressure range of from about 0.1 MPa (atmospheric pressure) to about 13.8 MPa is preferred. A pressure range of from about 1 to about 7 is further preferred.

The time for the reaction is determined empirically so as to maximize the production of levulinic acid esters at a selected temperature and pressure.

The levulinic acid ester products from the reaction of α-angelica lactone with olefins will comprise 10% to 100% of the total products formed, and may be cis- and/or trans-isomeric mixtures. Additional products, such as levulinic acid, may also be formed. Levulinic acid produced in the reaction may be converted in a separate step to α-angelica lactone by using vacuum distillation as described in U.S. Pat. No. 2,809,203; α-angelica lactone formed by this process may then be recycled back to Reaction (A) for the production of additional levulinic acid esters.

During formation of the esters in Reaction (A), the aqueous phase and organic phase can be processed in any desirable manner to recover the valuable products contained therein. The aqueous phase contains water, any inorganic acid or catalytic components that may be present, as well as a very small amount of the organic acids, which were not esterified. This aqueous layer is most suitably processed by subjecting to distillation to remove the water fraction. Any inorganic acid or catalytic components will then be in concentrated form in the residue and may be used as desired or discarded. However, any other suitable processing technique may be employed.

The organic phase will be found to contain the water-immiscible solvent and the esters, and may be processed as desired. A suitable processing technique is to subject the organic phase to distillation by fractionation so as to separate the water-immiscible solvent as one fraction and the mixed esters contained therein as second or separate fractions. This ester mixture can then be used as a fuel additive. If the organic solvent is such that it could be used as an additive to fuel, for example, iso-octane, separation of the esters may not be required.

The process of the invention as described is directed to the batch method of production. The process can also be performed by a continuous method using a combination of reaction and extraction procedures. The continuous method requires the use of a series of esterifiers and extraction tanks. Thereby, a given feed material may be continuously esterified and extracted with the esterifying olefin and extractant solvent being continuously recycled as they are recovered.

In the continuous method, the olefins are mixed in an esterification zone with the angelica lactone. Thereafter, this mixture is passed into the extraction zone where it is admixed with the water-immiscible extractant solvent, the mixing being preferably conducted under countercurrent conditions for good contact. In this zone, the extractant solvent continuously extracts the esters from the aqueous phase to the organic phase. The resulting mixture is then passed to a separation or settling zone where the layers are separated as an aqueous phase containing any water-soluble impurities, and an organic phase containing the esters in the extractant solvent. The aqueous phase can then be distilled to remove the excess olefin and the olefin is recycled to the esterification zone for further reaction with additional angelica lactone feed and makeup olefin. Any acid catalyst present may also be recovered and recycled to the esterification chamber. Moreover, the organic phase is subsequently distilled to remove the extractant solvent and the latter is recycled to the extraction chamber.

The process of the present invention may be carried out in batch, sequential batch (i.e., a series of batch reactors) or in continuous mode in any of the equipment customarily employed for continuous process (see for example, H. S. Fogler, Elementary Chemical Reaction Engineering, Prentice-Hall, Inc., New Jersey, USA).

α-Angelica lactone may be obtained by vacuum distilling levulinic acid as described in U.S. Pat. No. 2,809,203, herein incorporated by reference. According to this method, levulinic acid may be subjected to continuous distillation at a pot temperature of 150° C. to 175° C. under a pressure of 17 to 50 mm Hg. The distillate comprising α-angelica lactone and water is removed as fast as the distillate is formed, and water is removed from the distillate to yield α-angelica lactone. α-Angelica lactone for use as fuel additives may also be obtained via levulinic acid produced from biomass. For the conversion of biomass to levulinic acid, biomass is contacted with water and an acid catalyst in a train of one or more reactors, preferably under pressure at elevated temperature. This basic process with modifications is described, for example, in U.S. Pat. No. 5,608,105, U.S. Pat. No. 5,859,263, and U.S. Pat. No. 6,054,611. Generally, cellulose in the biomass is converted to levulinic acid and formate in one or more reactors. Mineral acids are generally used for hydrolysis at concentrations ranging from about 1% to about 50%; initial biomass concentrations range from about 1% to about 75%. The temperature is maintained at approximately 150° C. to about 300° C., usually thr ough the use of high pressure steam; pressure is required to maintain volatile components in solution. The time for the reaction can range from about 3 seconds to about 3 hours. Time, pressure, temperature and acid concentration vary according to the nature of the substrate biomass. Following hydrolysis, the levulinic acid is separated from the formic acid, generally through a reduction in pressure, and optionally temperature, to form a gaseous stream of aqueous formic acid.

In the acid hydrolysis of biomass, lactones may be formed in addition to levulinic acid. Generally, lactones have been considered undesirable by-products of the process. U.S. Pat. No. 3,258,481, for example, describes a process for removal of the lactones in order to obtain levulinic acid of high purity and light color. In the process described herein, lactones are a desirable end-product, and lactones formed in the acid hydrolysis remain in the levulinic acid stream.

An example of a process for hydrolyzing biomass to form levulinic acid comprises adding a dilute acid solution to biomass solids to make a slurry which comprises about 1% to about 50% biomass solids by weight, and preferably about 10% to about 30% by weight. The slurry is pumped into a reactor that is heated with high pressure steam to about 200° C. to about 250° C. Alternatively, the acid may be added as a separate feed to the reactor that contains the biomass. The biomass may be physically disrupted by, for example, crushing or grinding prior to acidification.

The acid used for the disruption of the biomass may be a dilute mineral acid, such as sulfuric acid, phosphoric acid, hydrochloric acid, nitric acid or a combination thereof, at a concentration of about 1% to about 10% acid by weight. The acid catalyst may also be any one of the alternative acid catalysts described above for the reaction of α-angelica lactone with olefins, used at about 1% to about 10% by weight.

The reaction pressure for disruption of the biomass is maintained at a level that prevents vaporization of the components, preferably at about 0.69 MPa to about 13.8 MPa; a reaction pressure of about 1 MPa to about 7 MPa is further preferred.

The time for the reaction is dependent on the acid concentration and the source of the biomass (for example wood chips, corn husks or paper sludge), as well as the temperature and pressure of the reactor and reactor size and type. Preferably the reaction time is about 5 seconds to about 120 min. It is well known to those skilled in the art that temperature, pressure and time for the reaction in a given reactor are interrelated. As temperature increases, the rate of hydrolysis of the biomass increases, however at high temperatures, the pressure will increase and the reaction may proceed too quickly and result in unwanted degradation products. Similarly, too high a concentration of acid can result in the formation of unwanted degradation products, as well as potentially resulting in equipment corrosion problems. Also, prolonged times can lead to unwanted degradation products. Thus, the temperature, pressure and time must be adjusted to achieve optimal degradation of the biomass.

The biomass generally comprises lignin, cellulose, starch, hemicellulose or combinations thereof. Cellulose and starch are converted to hexose oligomers and monomers, such as glucose. The hexose oligomers are further hydrolyzed to hexose monomers, and the hexose monomers are further converted to hydroxymethylfurfural, as well as other reaction intermediates. Hemicellulose, if present, is degraded to pentose and hexose oligomers and monomers. The hexose monomers are further hydrolyzed as above, and the pentose oligomers and monomers are further converted to furfural in this reaction.

The reaction mixture from this first reaction of biomass hydrolysis may then be pumped into a second reactor to complete the conversion of the biomass. In this second reactor conditions are adjusted so as to maximize the conversion of hydroxymethylfurfural to levulinic acid and formic acid. Remaining hexose monomers from the first reaction may be converted to hydroxymethylfurfural, and then to levulinic acid plus formic acid. The mineral acid concentration in this second reactor is preferably about 2% to about 10% by weight. The temperature of the reactor, maintained by high pressure steam, is about 150° C. to about 210° C. The time for the reaction is preferably 10 minutes to about 60 minutes; a time of 15 minutes to about 30 minutes is further preferred.

In the second reactor, furfural, if present, and formic acid vaporize; the vapor can be removed from the reactor and condensed back to furfural and formic acid. Additional reaction intermediates may also be vaporized and condense as part of the furfural/formic acid mixture. Solids, such as lignin, that are not solubilized at the end of the reaction can be removed by an appropriate separation technique, such as filtration. Other methods include sedimentation, centrifugation and decantation; combinations of solid-liquid separation techniques may also be used. Levulinic acid produced in the reaction can be removed as a liquid stream and recrystallized, if necessary. Alternatively, the levulinic acid can be removed from the solids by vacuum distillation. The levulinic acid is then available for use as a precursor for the synthesis of α-angelica lactone, as described above. The levulinic acid stream used for the synthesis of α-angelica lactone may comprise lactones produced in the hydrolysis of the biomass. The acid catalyst used in the reaction can be recycled for use within the process. α-Angelica lactone produced in the acid hydrolysis, or from the vacuum distillation of levulinic acid, can then be used according to Reaction (A) for the synthesis of levulinic acid esters.

It had previously been believed that in order to produce a superior fuel additive, the formic acid would have to be separated along with the solids from the reaction mixture to form pure levulinic acid as a fuel additive. Subsequently, the levulinic acid would be used for its intended purpose (see, for example, U.S. Pat. No. 5,608,105). However, the present invention teaches a process for obtaining a fuel additive derived from the mixture of α-angelica lactone and formic acid. According to this process, formic acid produced from the degradation of biomass is utilized for the production of fuel additives, allowing for a more complete utilization of the biomass starting material, and the mixture of levulinic acid and formic acid esters obtained as a result of a reaction with olefins will have superior oxygenate values compared to those of pure levulinic acid esters. For example, the oxygen content per unit weight of the 50/50 weight percent mixture of ethyl levulinate and ethyl formate is about 15% higher than that. of pure ethyl levulinate.

Thus, according to the process of the invention, furfural, if present, can be separated from the mixture of furfural and formic acid that is produced by condensation of the vapors from the second reactor described above by using azeotropic distillation. Alternatively, furfural can be separated from formic acid by, for example, chromatography, such as ion exchange, or liquid-liquid separation using an appropriate solvent.

The formic acid recovered by condensation, or from the separation of the components of the furfural/formic acid mixture, can then be added to α-angelica lactone for use in a reaction with olefins as described above for the reaction of α-angelica lactone with olefins.

Persons skilled in the art will recognize that any method of biomass hydrolysis can be utilized which produces levulinic acid and formic acid as products from the degradation of hexose monomers. In the process utilized, furfural may be produced from the degradation of pentose monomers. Furfural and formic acid are separated from the furfural/formic acid/levulinic acid mixture yielding levulinic acid, which can then be converted to α-angelica lactone. Furfural is then separated from the formic acid; formic acid is recovered and combined with α-angelica lactone for further conversion to the esters of levulinic and formic acids.

The reaction of α-angelica lactone plus formic acid with olefins produces a mixture of levulinic acid esters (II) and formic acid esters (III) as shown in the following Reaction (B): 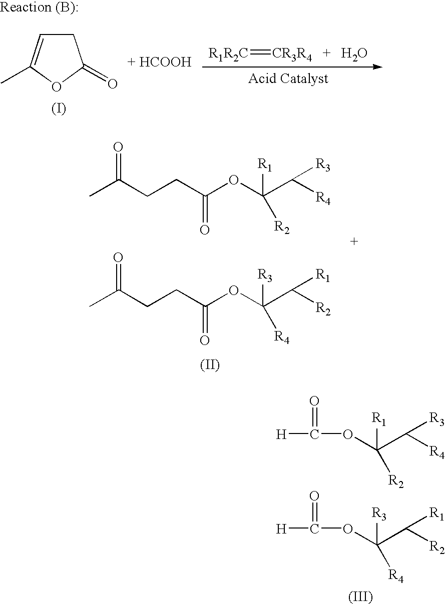
wherein R₁, R₂, R₃ and R₄ are independently selected from the group consisting of hydrogen, C₁-C₁₀ unsubstituted or substituted alkyl, C₁-C₁₀ unsubstituted or substituted alkenyl and C₃-C₂₀ unsubstituted or substituted cycloalkyl, wherein optionally any two or more of R₁, R₂, R₃, and R₄ can together form a cyclic or bicyclic alkyl group, wherein the unfilled valences in α-angelica lactone, the levulinic acid esters and the formic acid esters are hydrogen, and wherein the total number of carbons in the olefin is not more than twenty two.

Typical olefins useful for the reaction include ethylene, propylene, butene, pentene, hexene, cyclohexene, etc. Mixtures of olefins may also be used.

The reaction conditions, catalysts, concentrations, process steps and recovery steps for Reaction (B) are identical to those described above for Reaction (A) of α-angelica lactone with olefins. The levulinic acid ester and formic acid ester products from the reaction of α-angelica lactone plus formic acid with olefins will comprise 10% to 100% of the total products formed, and may be cis- and/or trans-isomeric mixtures. Additional products, such as levulinic acid, may also be formed.

Unreacted olefins used in excess in either Reaction (A) or Reaction (B) can be directly added to fuel without downgrading the fuel value as measured by octane number or cetane number.

As liquid organic based fuels for use in internal combustion engines, the reaction mixture containing levulinic acid esters or the mixture of levulinic acid and formic acid esters as obtained from the processes of the invention, can be used in the range of from about 1% to about 99% by volume, as additive to gasoline, diesel, or biofuel. A preferred range is from about 1% to about 90% by volume. A more preferred range is from about 1% to about 50% by volume. A further preferred range is from about 1% to about 20% by volume. Moreover, the reaction mixtures of esters of this invention can also be used as 100% fuel.

The mixture of esters prepared according to the processes of the invention contains significantly higher oxygen content than MTBE.

Therefore, a lesser amount of esters is required in order to meet the various clean fuel programs' oxygen requirements for gasoline. Also, the mixture of esters made by the processes of the invention in general has lower vapor pressures than MTBE. This indicates that the esters will also have a lower Reid Vapor Pressure (RVP) than MTBE. The addition to gasoline of esters that have low RVP is beneficial because it enables the addition of high RVP paraffins, such as butane, isobutane and isopentane, in order to meet the regulated RVP requirements of gasoline. Previously reported blending octane numbers show that the esters made by the process of the invention can also act as octane-enhancing agents for gasoline. Table 1 herein demonstrates comparative data for fuel additive characteristics of the reaction mixture of the invention as compared to MTBE. The information for Table 1 was obtained from the following references: [1] Design Institute for Physical Property Data (DIPPR)®, Project 801, Sponsor Release January 2002.

[2] Alternatives to Traditional Transportation Fuels: An Overview. Report DOE/EIA-0585/0 (June 1994). [3] Jungbluth et al. Liquid Fuels.

International Patent Application WO 94/21753 (1994).

TABLE 1
Fuel Additive Characteristics of Ester Oxygenates Made by the Process of the Invention-Comparative Data

| | Fuel Additive Characteristics | | | | | | | |
|---|---|---|---|---|---|---|---|---|
| | | | | | Vapor | Lower | Lower | Blending |
| | O₂ | 2 | 2.7 | Flash | Press. | Heating | Heating | Octan No |
| | Content | Wt. %* | Wt. %** | Point [1] | (100° F.) | Value [2] | Value [1] | R + M/2 |
| NAME | wt % | wt % | wt % | ° C. | [1] KPa | Btu/lb | Btu/lb | Reported |
| MTBE | 18 | 11 | 14.9 | −28.15 | 55 | 16578 | 15100[3] | 108[3] |
| Ethanol | 35 | 5.8 | 7.8 | 12.85 | 16 | 12477 | 11500[3] | 100[3] |
| Methanol | 50 | 4 | 5.4 | 10.85 | 32 | 8950 | 8570 | |
| Methyl Formate | 53 | 3.8 | 5.1 | −19.15 | 126.2 | 7711 | 6393 | 101.5[4] |
| Ethyl Formate | 43 | 4.6 | 6.3 | −20 | 55 | 10139 | 8751 | 102.5[4] |
| N-Propyl | 36 | 5.5 | 7.4 | −3.15 | 19.9 | 11792 | 9964 | |
| Formate | | | | | | | | |
| Isopropyl | 36 | 5.5 | 7.4 | −6 | | 12304 | | |
| Formate | | | | | | | | |
| N-Butyl Formate | 31 | 6.4 | 8.6 | 18 | 7.4 | 15175 | 11415 | |
| Isobutyl | 31 | 6.4 | 8.6 | 3.85 | 10.2 | 12797 | 11377 | |
| Formate | | | | | | | | |
| Sec-Butyl | 31 | 6.4 | 8.6 | 0.85 | 12.1 | 10617 | | |
| Formate | | | | | | | | |
| Tert-Butyl | 31 | 6.4 | 8.6 | −9 | 19.8 | 12873 | 11277 | |
| Formate | | | | | | | | |
| Amyl Formate | 28 | 7.3 | 9.8 | 26.85 | 2.5 | 13905 | 12274 | |
| Ester | | | | | | | | |
| Methyl | 37 | 5.4 | 7.3 | | | 10773 | | 106.5[4] |
| Levulinate | | | | | | | | |
| Ethyl Levulinate | 33 | 6.6 | 8.1 | | | 11722 | | 107.5[4] |
| N-Propyl | 30 | 6.6 | 8.9 | | | 13912 | | |
| Levulinate | | | | | | | | |
| Isopropyl | 30 | 6.6 | 8.9 | | | 10969 | | 105[4] |
| Levulinate | | | | | | | | |
| N-Butyl | 28 | 7.2 | 9.7 | | | 14451 | | |
| Levulinate | | | | | | | | |
| Isobutyl | 28 | 7.2 | 9.7 | | | 13041 | | 102.5[4] |
| Levulinate | | | | | | | | |
| Sec-Butyl | 28 | 7.2 | 9.7 | | | 11748 | | 102.5[4] |
| Levulinate | | | | | | | | |
| Tert-Butyl | 28 | 7.2 | 9.7 | | | 12010 | | |
| Levulinate | | | | | | | | |
| Amyl Levulinate | 26 | 7.8 | 10.5 | | | 14909 | | |
| Isoamyl | 26 | 7.8 | 10.5 | | | 13605 | | |
| Levulinate | | | | | | | | |

*amount in oxygenated gasoline (2 wt % oxygen)
**amount in oxygenated gasoline (2.7 wt % oxygen)

### References for Table 1

[1] Design Institute for Physical Property Data (DIPPR)®, Project 801, Sponsor Release January 2002

[2] Walters, R. N. Molar Group Contributions to the Heat of Combustion.

Report DOT/FAA/AR-TNo1/75 to the US Department of Transportation (September 2001)

[3] Alternatives to Traditional Transportation Fuels: An Overview. Report DOE/EIA-0585/O (June 1994)

[4] Jungbluth et al. Liquid Fuels. International Patent Application WO 94/21753 (1994)

### EXAMPLES

The invention is further demonstrated by the following Examples.

### Example 1

### General Procedure for the Reaction of Olefins with Water and α-Angelica Lactone

A 2 cc pressure vessel was charged with 700 mg of a solution consisting of olefin, α-angelica lactone and 50 mg of an acid catalyst. The reactor was pressurized with nitrogen and heated to reactor temperature for a specified period of time. The vessel was then cooled, vented and the products analyzed by gas chromatography on a HP-6890 GC and HP-5972A GC-MS detector equipped with 25M×0.25 MM ID CP-Wax 58(FFAP) column. The GC yields were obtained by adding methoxyethyl ether as the internal standard. A similar procedure would be used for the reaction of olefins with water, α-angelica lactone, and formic acid.

The examples described below were performed according to a similar procedure under the conditions indicated for each example. For those experiments wherein the catalyst was added wet, no additional water was added to the reaction.

### Examples 2-36

### Reaction of α-Angelica Lactone (AGL) with 1-Hexene (1-HxEN), trans-2-Hexene (trans-2-HxEN), or trans-3-Hexene (trans-3-HxEN) and Water

Abbreviations: HxLV, hexyl levulinate; LA, levulinic acid; DXN, dioxane

| | | | | | | H₂O/1- | | | |
|---|---|---|---|---|---|---|---|---|---|
| | | | | N₂ | | HxEN | AGL | HxLV | LA |
| Expt. | | | | Pressure | | mole | Conversion | Selectivity | Selectivity |
| No. | Catalyst | Time (hrs) | Temp (C.) | (MPa) | Feedstock | ratio | (%) | (%) | (%) |
| 2 | H₂SO₄ | 2 | 150 | 5.52 | 30% AGL/28% 1- | 1 | 83.99 | 4.00 | 42.89 |
| | | | | | HxEN/H₂O/DXN | | | | |
| 3 | H₂SO₄ | 2 | 150 | 5.52 | 19.0% AGL/81.0% 1- | 1 | 97.58 | 31.52 | 6.79 |
| | | | | | HxEN/H₂O | | | | |
| 4 | CBV-3020E | 2 | 150 | 5.52 | 19.0% AGL/81.0% 1- | 1 | 99.70 | 18.37 | 5.54 |
| | | | | | HxEN/H₂O | | | | |
| 5 | Amberlyst 15 | 2 | 150 | 5.52 | 19.0% AGL/81.0% 1- | 1 | 99.61 | 54.05 | 8.57 |
| | | | | | HxEN/H₂O | | | | |
| 6 | H₂SO₄ | 2 | 150 | 5.52 | 70.0% AGL/30.0% 1- | 1 | 78.33 | 6.90 | 24.05 |
| | | | | | HxEN/H₂O | | | | |
| 7 | CBV-3020E | 2 | 150 | 5.52 | 70.0% AGL/30.0% 1- | 1 | 83.02 | 8.03 | 56.40 |
| | | | | | HxEN/H₂O | | | | |
| 8 | Amberlyst 15 | 2 | 150 | 5.52 | 70.0% AGL/30.0% 1- | 1 | 79.89 | 21.19 | 72.92 |
| | | | | | HxEN/H₂O | | | | |
| 9 | Zn(BF₄)₂ | 2 | 150 | 5.52 | 19.0% AGL/81.0% 1- | 1 | 98.57 | 45.53 | 5.39 |
| | | | | | HxEN/H₂O | | | | |
| 10 | AgBF₄ | 2 | 150 | 5.52 | 19.0% AGL/81.0% 1- | 1 | 99.39 | 33.49 | 2.73 |
| | | | | | HxEN/H₂O | | | | |
| 11 | Zn(OTf)₂ | 2 | 150 | 5.52 | 19.0% AGL/81.0% 1- | 1 | 99.32 | 16.82 | 2.59 |
| | | | | | HxEN/H₂O | | | | |
| 12 | Zn(OTs)₂ | 2 | 150 | 5.52 | 19.0% AGL/81.0% 1- | 1 | 99.00 | 44.00 | 5.56 |
| | | | | | HxEN/H₂O | | | | |
| 13 | Zn(OAc)₂ | 2 | 150 | 5.52 | 19.0% AGL/81.0% 1- | 1 | 99.43 | 6.71 | 2.14 |
| | | | | | HxEN/H₂O | | | | |
| 14 | Amberlyst | 2 | 150 | 5.52 | 19.0% AGL/81.0% 1- | 0 | 98.79 | 59.02 | 7.61 |
| | 36(wet) | | | | HxEN | | | | |
| 15 | Amberlite IR- | 2 | 150 | 5.52 | 19.0% AGL/81.0% 1- | 0 | 99.07 | 51.63 | 7.45 |
| | 120(plus) | | | | HxEN | | | | |
| 16 | Amberlite IRP- | 2 | 150 | 5.52 | 19.0% AGL/81.0% 1- | 1 | 99.63 | 0.07 | 1.11 |
| | 69 | | | | HxEN/H₂O | | | | |
| 17 | H₂SO₄ | 2 | 120 | 5.52 | 19.0% AGL/81.0% 1- | 1 | 99.38 | 50.82 | 4.63 |
| | | | | | HxEN/H₂O | | | | |
| 18 | Amberlyst 15 | 2 | 120 | 5.52 | 19.0% AGL/81.0% 1- | 1 | 99.02 | 62.73 | 11.26 |
| | | | | | HxEN/H₂O | | | | |
| 19 | Amberlyst | 2 | 120 | 5.52 | 19.0% AGL/81.0% 1- | 0 | 98.31 | 83.14 | 12.41 |
| | 36(wet) | | | | HxEN | | | | |
| 20 | AmberliteIR- | 2 | 120 | 5.52 | 19.0% AGL/81.0% 1- | 0 | 99.17 | 36.93 | 5.74 |
| | 120 (plus) | | | | HxEN | | | | |
| 21 | Zn(BF₄)₂ | 2 | 120 | 5.52 | 19.0% AGL/81.0% 1- | 1 | 99.68 | 4.80 | 1.32 |
| | | | | | HxEN/H₂O | | | | |
| 22 | AgBF₄ | 2 | 120 | 5.52 | 19.0% AGL/81.0% 1- | 1 | 99.34 | 53.00 | 3.20 |
| | | | | | HxEN/H₂O | | | | |
| 23 | Zn(OTs)₂ | 2 | 120 | 5.52 | 19.0% AGL/81.0% 1- | 1 | 99.64 | 3.76 | 1.38 |
| | | | | | HxEN/H₂O | | | | |
| 24 | H₂SO₄ | 2 | 90 | 5.52 | 19.0% AGL/81.0% 1- | 1 | 98.99 | 67.23 | 3.47 |
| | | | | | HxEN/H₂O | | | | |
| 25 | Amberlyst 15 | 2 | 90 | 5.52 | 19.0% AGL/81.0% 1- | 1 | 99.73 | 4.76 | 1.46 |
| | | | | | HxEN/H₂O | | | | |
| 26 | Amberlyst 36 | 2 | 90 | 5.52 | 19.0% AGL/81.0% 1- | 0 | 99.31 | 2.23 | 4.90 |
| | (wet) | | | | HxEN | | | | |
| 27 | AmberliteIR- | 2 | 90 | 5.52 | 19.0% AGL/81.0% 1- | 0 | 99.51 | 9.45 | 2.89 |
| | 120 (plus) | | | | HxEN | | | | |
| 28 | Zn(BF₄)₂ | 2 | 90 | 5.52 | 19.0% AGL/81.0% 1- | 1 | 99.75 | 0.21 | 1.17 |
| | | | | | HxEN/H₂O | | | | |
| 29 | AgBF₄ | 2 | 90 | 5.52 | 19.0% AGL/81.0% 1- | 1 | 99.83 | 0.13 | 0.75 |
| | | | | | HxEN/H₂O | | | | |
| 30 | Zn(OTs)₂ | 2 | 90 | 5.52 | 19.0% AGL/81.0% 1- | 1 | 99.70 | 0.09 | 1.40 |
| | | | | | HxEN/H₂O | | | | |
| 31 | H₂SO₄ | 2 | 150 | 5.52 | 19.0% AGL/81.0% | 1 | 99.14 | 26.10 | 8.33 |
| | | | | | trans-2-HxEN/H₂O | | | | |
| 32 | CBV-3020E | 2 | 150 | 5.52 | 19.0% AGL/81.0% | 1 | 99.61 | 16.76 | 3.47 |
| | | | | | trans-2-HxEN/H₂O | | | | |
| 33 | Amberlyst 15 | 2 | 150 | 5.52 | 19.0% AGL/81.0% | 1 | 99.45 | 47.75 | 6.81 |
| | | | | | trans-2-HxEN/H₂O | | | | |
| 34 | H₂SO₄ | 2 | 150 | 5.52 | 19.0% AGL/81.0% | 1 | 99.60 | 23.74 | 3.38 |
| | | | | | trans-3-HxEN/H₂O | | | | |
| 35 | CBV-3020E | 2 | 150 | 5.52 | 19.0% AGL/81.0% | 1 | 99.72 | 8.59 | 1.93 |
| | | | | | trans-3-HxEN/H₂O | | | | |
| 36 | Amberlyst 15 | 2 | 150 | 5.52 | 19.0% AGL/81.0% | 1 | 99.49 | 45.26 | 6.31 |
| | | | | | trans-3-HxEN/H₂O | | | | |

### Example 37-42

### Reaction of α-Angelica Lactone with 2,3-Dimethylbutene (DMB) and Water

Abbreviations: DMBLV, dimethylbutyl levulinate; LA, levulinic acid.

| | | | | N₂ | | | AGL | DMBLV | LA |
|---|---|---|---|---|---|---|---|---|---|
| Expt. | | Time | Temp | Pressure | | H₂O/DMB | Conversion | Selectivity | Selectivity |
| No. | Catalyst | (hrs) | (C.) | (MPa) | Feedstock | mole ratio | (%) | (%) | (%) |
| 37 | H₂SO₄ | 2 | 120 | 5.52 | 18.9% | 1 | 100.00 | 6.82 | 6.04 |
| | | | | | AGL/81.1% | | | | |
| | | | | | DMB/H₂O | | | | |
| 38 | Amberlyst | 2 | 120 | 5.52 | 18.9% | 1 | 100.00 | 7.09 | 5.98 |
| | 15 | | | | AGL/81.1% | | | | |
| | | | | | DMB/H₂O | | | | |
| 39 | Amberlyst | 2 | 120 | 5.52 | 18.9% | 0 | 100.00 | 9.73 | 8.34 |
| | 36 (wet) | | | | AGL/81.1% | | | | |
| | | | | | DMB/H₂O | | | | |
| 40 | AmberliteI | 2 | 120 | 5.52 | 18.9% | 0 | 100.00 | 3.97 | 5.82 |
| | R-120 | | | | AGL/81.1% | | | | |
| | (plus) | | | | DMB/H₂O | | | | |
| 41 | AgBF₄ | 2 | 120 | 5.52 | 18.9% | 1 | 100.00 | 8.34 | 6.61 |
| | | | | | AGL/81.1% | | | | |
| | | | | | DMB/H₂O | | | | |
| 42 | Zn(OTs)₂ | 2 | 120 | 5.52 | 18.9% | 1 | 100.00 | 9.24 | 6.59 |
| | | | | | AGL/81.1% | | | | |
| | | | | | DMB/H₂O | | | | |

### Examples 43-49

### Reaction of α-Angelica Lactone with Cyclohexene (CyHxEN) and Water

Abbreviations: CyHxLV, cyclohexyl levulinate; LA, levulinic acid.

| | | | | | | H₂O/1- | AGL | CyHxLV | |
|---|---|---|---|---|---|---|---|---|---|
| Expt. | | Time | | N₂ | | CyHxEN | Conversion | Selectivity | LA |
| No. | Catalyst | (hrs) | Temp (C.) | Pressure (MPa) | Feedstock | mole ratio | (%) | (%) | Selectivity (%) |
| 43 | H₂SO₄ | 2 | 120 | 5.52 | 19.2% | 1 | 97.67 | 60.88 | 4.37 |
| | | | | | AGL/80.8% | | | | |
| | | | | | CyHxEN/H₂O | | | | |
| 44 | Amberlyst | 2 | 120 | 5.52 | 19.2% | 1 | 99.10 | 83.54 | 7.41 |
| | 15 | | | | AGL/80.8% | | | | |
| | | | | | CyHxEN/H₂O | | | | |
| 45 | Amberlyst | 2 | 120 | 5.52 | 19.2% | 0 | 99.15 | 78.22 | 7.31 |
| | 36 (wet) | | | | AGL/80.8% | | | | |
| | | | | | CyHxEN | | | | |
| 46 | AmberliteIR- | 2 | 120 | 5.52 | 19.2% | 0 | 98.98 | 32.33 | 9.82 |
| | 120 (plus) | | | | AGL/80.8% | | | | |
| | | | | | CyHxEN | | | | |
| 47 | Zn(BF₄)₂ | 2 | 120 | 5.52 | 19.2% | 1 | 99.43 | 20.98 | 5.83 |
| | | | | | AGL/80.8% | | | | |
| | | | | | CyHxEN/H₂O | | | | |
| 48 | Zn(BF₄)₂ | 2 | 120 | 5.52 | 19.2% | 1 | 98.51 | 86.93 | 5.94 |
| | | | | | AGL/80.8% | | | | |
| | | | | | CyHxEN/H₂O | | | | |
| 49 | Zn(OTs)₂ | 2 | 120 | 5.52 | 19.2% | 1 | 99.76 | 5.47 | 1.59 |
| | | | | | AGL/80.8% | | | | |
| | | | | | CyHxEN/H₂O | | | | |

### Examples 50-56

### Reaction of α-Angelica Lactone with Propylene and Water

Abbreviations: i-Pr, isopropyl; LA, levulinic acid.

| | | | | Propylene | N₂ | | AGL | i-PrLV | LA |
|---|---|---|---|---|---|---|---|---|---|
| | | Time | Temp | Pressure | Pressure | | Conversion | Selectivity | Selectivity |
| Expt. No. | Catalyst | (hrs) | (C.) | (MPa) | (MPa) | Feedstock | (%) | (%) | (%) |
| 50 | H₂SO₄ | 2 | 120 | 0.75 | 2.75 | 84.5% AGL/15.5% H₂O | 93.82 | 3.37 | 9.85 |
| 51 | Amberlyst | 2 | 120 | 0.75 | 2.75 | 84.5% AGL/15.5% H₂O | 98.54 | 7.08 | 20.84 |
| | 15 | | | | | | | | |
| 52 | Amberlyst | 2 | 120 | 0.75 | 2.75 | 84.5% AGL/15.5% H₂O | 99.11 | 12.66 | 45.22 |
| | 36(wet) | | | | | | | | |
| 53 | Amberlite | 2 | 120 | 0.75 | 2.75 | 84.5% AGL/15.5% H₂O | 76.84 | 3.62 | 47.03 |
| | IR-120(plus) | | | | | | | | |
| 54 | Zn(BF₄)₂ | 2 | 120 | 0.75 | 2.75 | 84.5% AGL/15.5% H₂O | 99.02 | 5.26 | 23.91 |
| 55 | Zn(BF₄)₂ | 2 | 120 | 0.75 | 2.75 | 84.5% AGL/15.5% H₂O | 99.24 | 3.83 | 14.10 |
| 56 | Zn(OTs)₂ | 2 | 120 | 0.75 | 2.75 | 84.5% AGL/15.5% H₂O | 53.86 | 0.26 | 60.71 |

### Examples 57-58

Reaction of α-Angelica Lactone and Formic Acid (FA) with Cyclohexene (CyHxEN) and Water

Abbreviations: CyHxFM, cyclohexylformate; LA, levulinic acid; Con, Conversion; Sel, Selectivity.

| | | | | N₂ | | H₂O/ | | | | | CyHxFM |
|---|---|---|---|---|---|---|---|---|---|---|---|
| Expt. | | Time | Temp | Pressure | | 1-HxEN | AGL | CyHxLV Sel. | LA Sel | FA Con. | Sel. |
| No. | Catalyst | (hrs) | (C.) | (MPa) | Feedstock | mole ratio | Con. (%) | (%) | (%) | (%) | (%) |
| 57 | H₂SO₄ | 2 | 120 | 5.52 | 15.8% AGL/8.7% | 1 | 100 | 83.82 | 8.22 | 100 | 82.11 |
| | | | | | FA/70.5% | | | | | | |
| | | | | | CyHxEN/5.0% | | | | | | |
| | | | | | H₂O | | | | | | |
| 58 | Amberlyst | 2 | 120 | 5.52 | 16.6% AGL/8.7% | 1 | 100 | 81.86 | 17.42 | 100 | 80.22 |
| | 15 | | | | FA/69.2% | | | | | | |
| | | | | | CyHxEN/5.5% | | | | | | |
| | | | | | H₂O | | | | | | |

### Examples 59-60

### Reaction of α-Angelica Lactone and Formic Acid (FA) with 1-Hexene (1-HxEN) and Water

Abbreviations: HXFM, hexylformate; LA, levulinic acid.

| | | | | N₂ | | H₂O/1- | AGL | HxLV | | | HxFM |
|---|---|---|---|---|---|---|---|---|---|---|---|
| Expt. | | Time | Temp | Pressure | | HxEN | Con. | Sel. | LA Sel | FA Con. | Sel. |
| No. | Catalyst | (hrs) | (C.) | (MPa) | Feedstock | mole ratio | (%) | (%) | (%) | (%) | (%) |
| 59 | H₂SO₄ | 2 | 120 | 5.52 | 19.8% AGL/7.8% FA/67.1%1- | 1 | 99.70 | 58.30 | 11.02 | 100.00 | 83.60 |
| | | | | | HxEN/5.3% H₂O | | | | | | |
| 60 | Amberlyst | 2 | 120 | 5.52 | 16.9% AGL/7.7% FA/69.9% | 1 | 99.60 | 88.14 | 7.38 | 100.00 | 51.85 |
| | 15 | | | | 1-HxEN/5.5% H₂O | | | | | | |

### Example 61

Reaction of α-Angelica Lactone and Formic Acid (FA) with 1-Hexene (1-HxEN), Cyclohexene (CyHxEN) and Water

Abbreviations: HxLV, hexyl levulinate; CyHxLV, cyclohexyl levulinate; LA, levulinic acid; HxFM, hexylformate; CyHxFM, cyclohexylformate.

| | | | | N₂ | | H₂O/ | AGL | | CyHxLV | | | HxFM | CyHxFM |
|---|---|---|---|---|---|---|---|---|---|---|---|---|---|
| Expt | | Time | Temp | Pressure | | 1-HxEN | Con. | HxLV Sel. | Sel. | LA | FA | Sel. | Sel. |
| No. | Catalyst | (hrs) | (C.) | (MPa) | Feedstock | mole ratio | (%) | (%) | (%) | Sel. (%) | Con. (%) | (%) | (%) |
| 61 | Amberlyst | 1 | 150 | 5.52 | 16.8% AGL/8.8% | 1 | 100 | 18.64 | 28.95 | 21.48 | 100.00 | 15.36 | 25.45 |
| | 15 | | | | FA/35.5% 1- | | | | | | | | |
| | | | | | HxEN/35.5% | | | | | | | | |
| | | | | | CyHxEN/3.3% H₂O | | | | | | | | |

### Example 62

### Preparation of Formate and Levulinate Esters From Biomass and Olefins

An aqueous solution of paper mill waste (5% by weight of non-bleached Kraft paper sludge) is hydrolyzed with sulfuric acid (3%) in a train of two reactors. The temperature in the first reactor is maintained at 210° C. to 230° C. with high pressure steam, and the paper sludge is hydrolyzed for 30 to 60 seconds. Time, pressure and temperature are adjusted to optimize conversion of the sludge to hydroxymethylfurfural. The hydrolyzed paper mill waste is transferred to a second reactor at reduced pressure and temperature (200° C.) for 15 to 30 minutes, and a gaseous stream of aqueous formic acid is removed overhead. The condensed phase containing levulinic acid and lactones is sent to a lactone reactor where α-angelica lactone is produced by vacuum distillation of the levulinic acid. The α-angelica lactone may then be reacted with olefins and water in the presence of a catalyst as described, for example, in Examples 2-56 to produce levulinic acid esters. Alternatively the α-angelica lactone may be combined with the aqueous formic acid stream and reacted with olefins in the presence of a catalyst as described, for example, in Examples 57-60 to produce levulinate and formate esters.

1. A process for preparing levulinic acid esters (II) from α-angelica lactone, the process comprising the steps of:
(a) contacting α-angelica lactone (I) with at least one olefin in the presence of water and an acid catalyst, optionally in the presence of a water immiscible solvent: 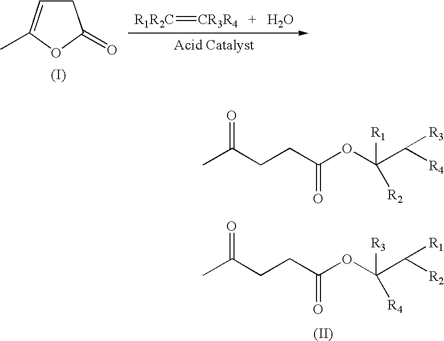
wherein:
(i) R₁, R₂, R₃ and R₄ are independently selected from the group consisting of hydrogen, C₁-C₁₀ unsubstituted or substituted alkyl, C₁-C₁₀ unsubstituted or substituted alkenyl and C₃-C₂₀ unsubstituted or substituted cycloalkyl, wherein optionally any two or more of R₁, R₂, R₃, and R₄ can together form a cyclic or bicyclic alkyl group, wherein the unfilled valences in α-angelica lactone and the levulinic acid esters are hydrogen, and wherein the total number of carbons in the olefin is not more than twenty two; and
(ii) the levulinic acid esters (II) comprise 10% to 100% of the products formed; and
(b) optionally recovering said levulinic acid esters.

2. A process for preparing a mixture of levulinic acid esters (II) and formic acid esters (III), the process comprising the steps of:
(a) contacting a mixture of α-angelica lactone (I) and formic acid with at least one olefin in the presence of water and an acid catalyst, optionally in the presence of a water immiscible solvent: 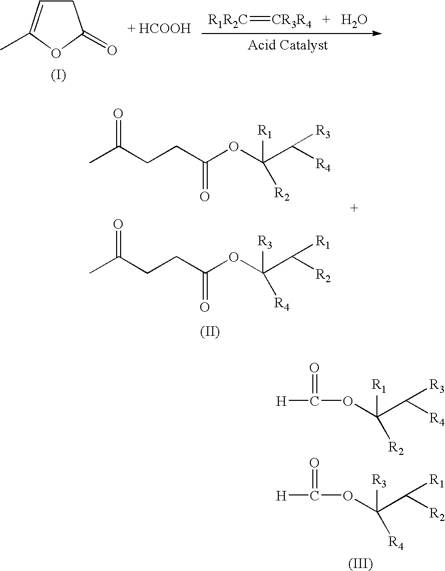
wherein:
(i) R₁, R₂, R₃ and R₄ are independently selected from the group consisting of hydrogen, C₁-C₁₀ unsubstituted or substituted alkyl, C₁-C₁₀unsubstituted or substituted alkenyl and C₃-C₂₀ unsubstituted or substituted cycloalkyl, wherein optionally any two or more of R₁, R₂, R₃, and R₄ can together form a cyclic or bicyclic alkyl group, wherein the unfilled valences in α-angelica lactone, the levulinic acid esters and the formic acid esters are hydrogen, and wherein the total number of carbons in the olefin is not more than twenty two; and
(ii) the levulinic acid esters (II) and formic acid esters (III) together comprise 10% to 100% of the products formed; and
(b) optionally recovering said levulinic acid esters and formic acid esters.

3. The process of claim 1 or claim 2 wherein said α-angelica lactone, or said mixture of α-angelica lactone and formic acid, are obtained from biomass, the process comprising:
(a) contacting biomass with water in the presence of an acid catalyst in one or more reactors, wherein:
(i) the initial biomass concentration comprises about 1 % to about 50% biomass solids by weight,
(ii) the acid catalyst comprises about 1% to about 30% acid by weight,
(iii) the pressure of the reaction is about 0.69 MPa to about 13.8 MPa,
(iv) the temperature of the reaction is about 150° C to about 300° C., and
(v) the time for the reaction is about 3 seconds to about 180 minutes;
to form a first reaction mixture comprising levulinic acid, formic acid and solids;
(b) reducing the pressure of said first reaction mixture such that an aqueous stream of gaseous formic acid is produced to form a second reaction mixture comprising levulinic acid and solids;
(c) condensing said gaseous formic acid produced in step (b) to form a third reaction mixture comprising formic acid;
(d) optionally removing said solids from said second reaction mixture and recovering said levulinic acid;
(e) converting said levulinic acid recovered in step (d) to α-angelica lactone by using vacuum distillation to form a fourth reaction mixture;
(f) optionally converting said α-angelica lactone in said fourth reaction mixture to levulinic acid esters according to the process of claim 1;
(g) optionally contacting said fourth reaction mixture with said third reaction mixture to form a sixth reaction mixture;
(h) contacting said sixth reaction mixture with at least one olefin according to the process of claim 2 to produce a mixture of levulinic acid esters and formic acid esters; and
(i) optionally recovering said levulinic acid esters produced in step (f) or said mixture of levulinic acid esters and formic acid esters produced in step (h).

4. The process of claim 1 or claim 2 wherein said α-angelica lactone, or said mixture of α-angelica lactone and formic acid, are obtained from biomass, the process comprising:
(a) contacting biomass with water in the presence of an acid catalyst in a reactor, wherein:
(i) the initial biomass concentration comprises about 1% to about 50% biomass solids by weight,
(ii) the acid catalyst comprises about 1% to about 10% acid by weight,
(iii) the pressure of the reaction is about 0.69 MPa to about 13.8 MPa,
(iv) the temperature of the reaction is about 200° C. to about 250° C., and
(v) the time for the reaction is about 5 seconds to about 120 minutes;
to form a first reaction mixture comprising hydroxymethylfurfural, optionally furfural and other reaction intermediates, and solids;
(b) optionally transferring said first reaction mixture to a second reactor wherein:
(vi)the acid concentration is from about 2% to about 10%,
(vii) the temperature of the reaction is from about 150° C. to about 210° C., and
(viii) the time for the reaction is from about 10 minutes to about 60 minutes;
to form a second reaction mixture comprising levulinic acid, formic acid, optionally furfural and other reaction intermediates, and solids;
(c) removing said formic acid and optionally said furfural from said second reaction mixture by condensation to form a third reaction mixture comprising formic acid and optionally furfural and a fourth reaction mixture comprising levulinic acid, optionally other reaction intermediates, and solids;
(d) optionally removing said solids from said fourth reaction mixture and recovering said levulinic acid;
(e) converting said levulinic acid to α-angelica lactone by using vacuum distillation to form a fifth reaction mixture;
(f) optionally converting said α-angelica lactone in said fifth reaction mixture to levulinic acid esters according to the process of claim 1;
(g) optionally removing furfural from said third reaction mixture by distillation or liquid-liquid separation to form a sixth reaction mixture comprising formic acid;
(h) optionally combining said fifth reaction mixture with said third reaction mixture or said sixth reaction mixture to form a seventh reaction mixture comprising α-angelica lactone and formic acid;
(i) contacting said seventh reaction mixture with at least one olefin according to the process of claim 2 to produce a mixture of levulinic acid esters and formic acid esters; and
(j) optionally recovering said levulinic acid esters produced in step (f) or said mixture of levulinic acid esters and formic acid esters produced in step (i).

5. The process of claim 1 or claim 2 wherein said acid catalyst, or metal salt thereof, has a pKa less than 4.

6. The process of claim 1 or claim 2 wherein said acid catalyst, or metal salt thereof, has a pKa less than 2.

7. The process of claim 1 or claim 2 wherein the acid catalyst is a heterogeneous acid catalyst having an Ho of less than or equal to 2.

8. The process of claims 1, 2, 3 or 4 wherein the acid catalyst is selected from the group consisting of inorganic acids, organic sulfonic acids, heteropolyacids, perfluoroalkyl sulfonic acids, metal salts thereof, mixtures of metal salts, and combinations thereof.

9. The process of claim 1 or claim 2 wherein the acid catalyst is selected from the group consisting of zeolites; CV-3020 zeolite; fluorinated alumina; acid-treated silica; acid-treated silica-alumina; acid-treated titania; acid-treated zirconia; heteropolyacids supported on zirconia, titania, alumina, silica; and combinations thereof.

10. The process of claim 1 or claim 2 wherein the acid is selected from the group consisting of metal sulfonates, metal sulfates, metal trifluoroacetates, metal triflates, and mixtures thereof; mixtures of salts with their conjugate acids, zinc tetrafluoroborate, and combinations thereof.

11. The process of claim 1 or claim 2 wherein the acid catalyst is selected from the group consisting of sulfuric acid, fluorosulfuric acid, phosphoric acid, p-toluenesulfonic acid, benzenesulfonic acid, phosphotungtstic acid, phosphomolybdic acid, trifluromethanesulfonic acid, 1,1,2,2-tetrafluorethanesulfonic acid, 1,1,1,2,3,4-hexafluorpropanesulfonic acid, bismuth triflate, yttrium triflate, ytterbium triflate, neodymium triflate, lanthanum triflate, scandium triflate, zirconium triflate, and combinations thereof.

12. The process of claim 1 or claim 2 wherein the temperature of the reaction is from about 0° C. to about 300° C.

13. The process of claim 1 or claim 2 wherein the pressure of the reaction is from about 0.1 MPa to about 13.8 MPa.

14. The process of claim 1 or claim 2 wherein the molar ratio of water to α-angelica lactone is from about 100 to 0.05.

15. The process of claim 1 or claim 2 wherein the molar ratio of water to α-angelica lactone is about 1 to 1.

16. The process of claim 1 or claim 2 wherein levulinic acid is produced.

17. The process of claim 16 wherein said levulinic acid is converted to α-angelica lactone, and wherein said α-angelica lactone is recycled back to the reaction of claim 1 or claim 2.

18. The process as recited in claim 1 or claim 2, wherein the total number of carbons in said olefin is not greater than 10, wherein the acid concentration is from about 0.25% to about 5%, wherein the temperature of the reaction is from about 100° C. to about 200° C., wherein the pressure of the reaction is from about 1 to about 7 MPa, and wherein the ratio of water to α-angelica lactone is about 1 to 1.

19. A composition comprising levulinic acid esters made by a process selected from the group of processes of claim 1, claim 2, claim 3 and claim 4.

20. The composition of claim 19 used as a fuel, an oxygenate for gasoline, an octane number-enhancing agent for gasoline, an oxygenate for diesel, a cetane number-enhancing agent for diesel or a fuel additive for biofuel.

21. A gasoline, diesel or biofuel comprising from 1% to 90% by volume of the composition of claims 19.

22. A gasoline, diesel or biofuel comprising from 1% to 50% by volume of the composition of claim 19.

23. A gasoline, diesel or biofuel comprising from 1% to 20% by volume of the composition of claims 19.

24. A process for manufacturing a fuel additive, the process comprising the process of claim 1 or claim 2.

25. A process for manufacturing a gasoline, a diesel fuel or a biofuel, the process comprising the process of claim 1 or claim 2.
